(19) United States
(12) Patent Application Publication
(10) Pub. No.: US 2007/0098731 A1
(43) Pub. Date: May 3, 2007

(54) Treatment of infection due to clostridium difficile

(75) Inventors: James Peter Burnie, Alderly Edge Cheshire (GB); Ruth Christine Matthews, Cheshire (GB)

Correspondence Address: ROTHWELL, FIGG, ERNST & MANBECK, P.C., 1425 K STREET, N.W., SUITE 800, WASHINGTON, DC, 20005, US

(73) Assignee: NeuTec Pharma PLC, Manchester Lancashire (GB)

(21) Appl. No.: 10/550,410

(22) PCT Filed: Mar. 25, 2004
(86) PCT No.: PCT/GB04/01383

§ 371 Date: Jun. 23, 2006

(30) Foreign Application Priority Data
Mar. 25, 2003 (GB) 0306782.4

(51) Int. Cl. A61K 39/40; C07H 21/04; C12P 21/06; C12N 1/21; C12N 15/74; C12P 21/08; C07K 16/12

(52) U.S. Cl. 424/167.1; 530/388.4; 435/69.1; 435/189; 435/252.3; 435/471; 536/23.2

(57) ABSTRACT

The present invention discloses a Clostridium difficile lactate dehydrogenase comprising the amino acid sequence of SEQ ID NO: 2, or an amino acid sequence exhibiting at least 70, 80, 90, 95, 96, 97, 98, 99, or 99.5% identity with the amino acid sequence of SEQ ID NO: 2. A Clostridium difficile lactate dehydrogenase comprising the amino acid sequence of SEQ ID NO: 2. Also disclosed are nucleic acid sequences encoding same, vectors and host cells, antibodies against same, medicaments and methods of manufacture of a medicament for the treatment of a Clostridium difficile infection, and diagnostic test kits and diagnostic test methods for same.

Clostridium difficile is a gram-positive anaerobic bacterium, and is deemed a significant human pathogen causing a spectrum of diseases ranging from mild diarrhoea to fulminant pseudomembranous colitis (PMC)—collectively referred to as C. difficile antibiotic-associated diarrhoea (CDAD). CDAD is a common, iatrogenic, nosocomial disease associated with substantial morbidity and mortality, especially in the elderly. Two factors have been assigned main roles in the pathogenesis of CDAD—the suppression of the resident intestinal flora by the administration of antibiotics, and the production by the bacterium of two high molecular weight toxins, toxin A and toxin B.

The bacterium is endemic in hospitals, and studies have shown that approximately one third of patients receiving antibiotic treatment in acute-care medical wards were colonised by C. difficile while in hospital (Kyne, L., et al., 2002, Clin. Infect. Dis. 34(3), pp346-53, PMID: 11774082). Of these patients, over half went on to develop CDAD while the remainder were symptomless carriers. CDAD is a major factor in extension of patient hospital stay times, and estimates suggest that the cost of this disease in the US exceeds $1.1 billion per year (Kyne, L., et al., Supra). Patients suffering from CDAD respond well to a treatment which includes a discontinuation of the inciting antibiotic and treatment with either of the antibiotics metronidazole and vancomycin. However, the use of e.g. vancomycin is one of last resort since it is associated with several problems. Not only may it cause nephrotoxicity, ototoxicity, bone marrow toxicity and the red man syndrome, but the problem with this treatment regime is that the CDAD often returns after successful treatment of the initial episode, and this reoccurrence represents a serious clinical problem. Additionally, there is evidence that C. difficile is becoming resistant to metronidazole and partially resistant to vancomycin, demonstrating the need for new alternatives in the treatment of CDAD.

Exotoxins A and B which are produced by pathogenic strains of the bacterium are cytotoxic, enterotoxic and proinflammatory, and are considered to be the main virulence factors of this non-invasive microorganism. However, not all infections with toxigenic strains result in disease, prompting the search for additional virulence factors. Bacterial surface expressed antigens represent candidate virulence factors, and are also considered important since such proteins likely mediate the essential functions such as adhesion to the epithelial layer of the gut in the first step of colonization or interaction with mediators of local immunity. In common with many other bacteria, C. difficile expresses a crystalline or paracrystalline surface layer (S-layer) on the outer cell surface. Such S-layers comprise proteins or glycoproteins forming a regularly arranged lattice on the external surface of the bacterium, and have previously been shown to be essential for the virulence of pathogens such as Aeromanas salmonicida and Campylobacter fetus. In contrast to most bacteria which comprise one S-layer, C. difficile is known to comprise two superimposed paracrystalline S-layers, each composed of a glycoprotein subunit which varies slightly in apparent molecular weight among different C. difficile strains. Most strains of C. difficile express two major S-layer proteins (SLPs), one of 32-38 kDa (low-MW SLP) and a second of 42-48 kDa (high-MW SLP). The low-MW SLP appears to be immunodominant and is the antigen most commonly recognised by patients suffering from CDAD, and is the only antigen recognised in EDTA extracts of bacteria by antisera raised in rabbits against whole C. difficile cells (Calabi, E. et al., 2001, Mol. Microbiol., 40(5) p1187-99, PMID: 11401722).

Other non-antibiotic based therapeutic regimes for the treatment/prevention of C. difficile infection are based upon passive immunization and vaccination. Passive immunization relies on the administration to a patient of toxin neutralising antibodies (as disclosed in WO 99/20304), or antibodies raised against the whole bacterium and the toxins (as disclosed in WO 96/07430). Vaccination treatment comprises administering to a patient either a nucleic acid sequence encoding an immunogenic fragment of the C. difficile surface layer protein or variant or homologue thereof, or an equivalent polypeptide fragment (as disclosed in WO 02/062379).

The present inventors have discovered that the low-MW SLP masks a previously unidentified bacterial protein of 36 kDa (as determined by SDS-PAGE and Western blotting of bacterial extracts probed with patient antisera). The fact that this protein is recognised by antibodies present within sera isolated from patients infected with Clostridium difficile potentially implicates the protein in the pathogenesis of CDAD. As such, this protein and epitopes thereof potentially offer a wide range of uses—they may be used therapeutically as immunogens, for example as vaccines, or diagnostically to detect agents (e.g. antibodies) which bind specifically to them. They may also be used to produce neutralising agents, for example antibodies, which may be used both therapeutically and diagnostically, either on their own, or in combination e.g. with other antibodies used for passive immunization.

According to the present invention there is provided a C. difficile lactate dehydrogenase (LDH) comprising the amino acid sequence of SEQ ID NO: 2. Some variation may exist within the amino acid sequence in accordance with the inter-strain variation exhibited by C. difficile. For example, a protein according to the present invention may exhibit at least 70, 80, 90, 95, 96, 97, 98, 99, or 99.5% identity with the amino acid sequence of SEQ ID NO: 2.

Sequence homology/identity is as determined using the BLAST2 program (Tatusova T A et al., FEMS Microbiol Lett. 1999 May 15;174(2):247-50; PMD: 10339815) at the National Center for Biotechnology Information, USA (www.ncbi.nlm.nih.gov) with default parameters. As used herein amino acid or nucleic acid “identity” is equivalent to amino acid or nucleic acid “homology”.

Also provided according to the present invention is an isolated nucleic acid molecule encoding the C. difficile LDH of the present invention.

For example the isolated nucleic acid molecule may comprise the nucleotide sequence of SEQ ID NO: 1. Some variation may exist within the sequence in accordance with e.g. inter-stain variation exhibited by C. difficile. For example, the sequence may exhibit at least 80, 85, 90, 95, 96, 97, 98, 99, or 99.5% identity with the nucleotide sequence of SEQ ID NO: 1.

Also provided according to the present invention is a nucleic acid vector comprising the nucleic acid molecule encoding the C. difficile LDH of the present invention. The term “vector” refers to a vehicle, preferably a nucleic acid molecule, which can transport the nucleic acid molecules. When the vector is a nucleic acid molecule, the nucleic acid molecules are covalently linked to the vector nucleic acid. With this aspect of the invention, the vector can be e.g. a plasmid, single or double stranded phage, a single or double stranded RNA or DNA viral vector, or artificial chromosome, such as a BAC, PAC, YAC, or MAC. The vector may be a cloning vector or an expression vector, and may function in prokaryotic or eukaryotic cells or in both (e.g. as a shuttle vector). Appropriate cloning, expression and shuttle vectors for prokaryotic and eukaryotic hosts are described in Sambrook, J. and Russell, D., “Molecular Cloning: A Laboratory Manual”, Third Edition, Cold Spring Harbor Laboratory, Cold Spring Harbor Press, New York, 2001.

Also provided according to the present invention is a host cell containing the vector encoding the C. difficile LDH of the present invention. A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

Also provided according to the present invention is a process for producing a polypeptide comprising the C. difficile LDH of the present invention, the process comprising culturing a host cell under conditions sufficient for the production of said polypeptide, and recovering said polypeptide.

The LDH protein or peptide sequences derived from it may be expressed and purified from cells that have been altered to express it (recombinant), or synthesized using known protein synthesis methods. For example, a nucleic acid molecule encoding the protein, or peptide sequence, is cloned into an expression vector, the expression vector introduced into a host cell and the protein expressed in the host cell. The protein can then be isolated from the cells by an appropriate purification scheme using standard protein purification techniques.

Also provided according to the present invention is a vector encoding the C. difficile LDH of the present invention, or an antigenic fragment thereof, wherein the isolated nucleic acid molecule is inserted into the vector in proper orientation and correct reading frame such that a polypeptide comprising the C. difficile LDH of the present invention, or an antigenic fragment thereof may be expressed by a cell transformed with said vector.

The isolated nucleic acid molecule as herein described may be operatively linked to a promoter sequence.

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor can be produced from the vector itself. It should be understood, however, that in some embodiments, transcription and/or translation of the nucleic acid molecules can occur in a cell-free system.

The regulatory sequences to which the nucleic acid molecules described herein can be operably linked includes promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook, J. and Russell, D., “Molecular Cloning: A Laboratory Manual”, Third Edition, Cold Spring Harbor Laboratory, Cold Spring Harbor Press, New York, 2001.

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules can be inserted into the vector nucleic acid by well-known methodologies. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art. The vector containing the appropriate nucleic acid molecule can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, plant cells, and human cells, such as HeLa, or HuH-7.

Also provided according to the present invention is an antibody or an antigen-binding fragment thereof specific against a C. difficile LDH of the present invention. For example, the antibody or an antigen-binding fragment thereof may be specific against the C. difficile LDH consisting the sequence of SEQ ID NO: 2.

Antibodies are well known (Harlow, E. and Lane, D., “Using Antibodies—A Laboratory Manual”, Cold Spring Harbor Laboratory, Cold Spring Harbor Press, New York, 1998). The term “antibody” in its various grammatical forms is used herein to refer to immunoglobulin molecules and immunologically active portions of immunoglobulin molecules, i.e., molecules that contain an antibody combining site or paratope. Such molecules are also referred to as “antigen binding fragments” of immunoglobulin molecules.

Illustrative antibody molecules are intact immunoglobulin molecules, substantially intact immunoglobulin molecules and those portions of an immunoglobulin molecule that contain the paratope, including those portions known in the art as Fab, Fab′, F(ab′)2, scFv and F(v). A binding agent according to the present invention may not only bind to the LDH but may also neutralise or ameliorate the bacterial infection.

The antibody or antigen-binding fragment thereof specific against a C. difficile LDH according to the present invention may be for use in a method of treatment or diagnosis of the human or animal body.

Also provided according to the present invention is a method of manufacture of a medicament for the treatment of a C. difficile infection, comprising the use of an antibody or an antigen-binding fragment thereof specific against a C. difficile LDH according to the present invention.

The LDH protein or epitopes derived from the LDH protein may be used to generate antibodies. Epitopes of the bacterial LDH protein may be determined using standard procedures (e.g. Geysen, H. M., et al., J. Immunol. Methods, 1987, 102(2):259-74, PMID: 2443575; Geysen, H. M., et al., J Mol. Recognit., 1988, 1(1):32-41, PMID: 2483922; Jung, G., and Beck-Sickinger, A. G., 1992, Angew. Chem. Int. Ed. Eng., 31:367486; Beck-Sickinger, A. G., Jung, G., Pharm. Acta. Helv. 1993;68(1):3-20; PMID: 7692453). Antibodies or antigen-binding fragments thereof may be prepared using established immunological techniques which are well-known to a person skilled in the art (see e.g. Harlow, E. and Lane, D., “Using Antibodies—A Laboratory Manual”, Cold Spring Harbor Laboratory, Cold Spring Harbor Press, New York, 1998). Thus, for example, any suitable host may be injected with the protein and the blood collected to yield the desired polyclonal antibody after appropriate purification and/or concentration (for example by affinity chromatography using the immobilised protein as the affinity medium). Alternatively splenocytes or lymphocytes may be recovered from the protein-injected host and immortalised using for example the method of Kohler et al. (1976, Eur. J. Immunol., 6: 511), the resulting cells being segregated to obtain a single genetic line producing monoclonal antibodies. Antibody fragments may be produced using conventional techniques, for example, by enzymatic digestion with pepsin or papain. Where it is desired to produce recombinant antibodies according to the invention these may be produced using, for example, the methods described in EP 171469, EP 173494, EP 194276 and EP 239400.

The medicament may be a vaccine. Vaccination protects against infection by priming the immune system with pathogen-derived antigen(s). Vaccination is effected by a single or repeated exposures to the pathogen-derived antigen(s) and allows antibody maturation and B cell clonal expansion without the deleterious effects of the full-blown infectious process. The LDH protein or epitopes derived from the protein may be used within a vaccine. A vaccine comprising one or more of such epitopes may be used to immunize a person, thereby decreasing the susceptibility to C. difficile infection. The protein and/or epitopes may be immunogenic, or they may be administered as an immunogenic composition for example comprising an adjuvant. The vaccine may be a DNA vaccine. DNA vaccines are well established and will be known to a person skilled in the art.

Since there is evidence in bacteria such as Enterococcus faecium (Bugg, T. D. et al., 1999, Biochemistry, 30(43), pp10408-15, PMID: 1931965) and Staphylococcus aureus (Milewski, W. M. et al., 1996, Antimicrob Agents Chemother 40(1), pp166-72, PMID: 8787900) that lactate dehydrogenases play an important role in the mechanism of resistance to antibiotics such as vancomycin, the combination of an antibody or an antigen-binding fragment thereof specific against a C. difficile LDH according to the present invention, together with an antibiotic e.g. a glycopeptide antibiotic such as vancomycin may represent a means by which an infection due to C. difficile may be more successfully treated. Alternatively, the glycopeptide antibiotic may be e.g. ramoplanin or teicoplanin. The antibiotic may be metronidazole, which is sometimes used in the treatment of CDAD.

The medicament may be used for the treatment of a C. difficile infection, and may comprise a therapeutically effective quantity of the antibody or an antigen-binding fragment thereof specific against a C. difficile LDH according to the present invention, or the same in combination with a glycopeptide antibiotic e.g. vancomycin, or an antibiotic such as metronidazole. The antibody or the antigen-binding fragment thereof may act synergistically with the antibiotic, providing an enhanced therapeutic effect, i.e. an effect greater than would be expected with treatment using the combination therapy.

The medicament may be used in a method of treatment or diagnosis of the human or animal body.

The medicament may be used for the treatment of a C. difficile infection, wherein the bacterium may be resistant to glycopeptide antibiotic treatment or antibiotic treatment.

Also provided according to the present invention is a method of manufacture of a medicament for the treatment of a C. difficile infection, characterised in the use of a therapeutically effective quantity of an antibiotic e.g. a glycopeptide antibiotic, and an antibody or an antigen-binding fragment thereof specific against a C. difficile LDH according to the present invention. The antibiotic may be a glycopeptide antibiotic such as vancomycin, ramoplanin or teicoplanin. Alternatively, the antibiotic may be metronidazole.

Also provided according to the present invention is a method of treatment of C. difficile infection, comprising the step of administering to a patient in need of same a therapeutically effective quantity of an antibiotic e.g. a glycopeptide antibiotic, and an antibody or an antigen-binding fragment thereof specific against a C. difficile LDH according to the present invention. The antibiotic may be a glycopeptide antibiotic such as vancomycin, ramoplanin or teicoplanin. Alternatively, the antibiotic may be metronidazole.

Medicaments and methods of treatment according to the present invention will be readily apparent to one skilled in the art. Medicaments may be prepared using pharmaceutically acceptable carriers, diluents or excipients (Remington's: The Science and Practice of Pharmacy (1995)Mack Publishing Company, Easton, Pa., USA). The medicaments and methods of treatment may be effected using a pharmaceutically effective amount of the epitope or antibody/antigen-binding fragment. Appropriate dosages will be readily apparent to one skilled in the art and may be readily determined, for example by means of dose-response experiments

Also provided according to the present invention is a diagnostic test method for detecting the presence in a sample of said C. difficile LDH, comprising the steps of:
i) contacting said sample with an antibody or an antigen-binding fragment thereof specific against a C. difficile LDH according to the present invention;
ii) detecting any antibody-antigen binding reaction; and
iii) correlating the results of detection step (ii) with the presence of said C. difficile LDH in said sample.

Also provided according to the present invention is a diagnostic test method for detecting the presence in a sample of antibody specific against a C. difficile LDH according to the resent invention, comprising the steps of:
i) contacting said C. difficile LDH with said sample;
ii) detecting any antibody-antigen binding reaction; and
iii) correlating the results of detection step (ii) with the presence of antibody specific against said C. difficile LDH in said sample.

The sample may be from a patient, for example a serum sample or a peritoneal dialysate, although any other sample which may contain, or which might be expected to contain, anti-C. difficile antibodies may of course be used.

Also provided according to the present invention is a diagnostic test kit for performing a diagnostic test method according to the present invention. Diagnostic test kits are well known and may for example include dip-stick tests according to WO 88/08534. The test kit may include instructions for its use in a diagnostic test method according to the present invention.

Also provided according to the present invention is a pharmaceutical pack for the treatment of a C. difficile infection, comprising a therapeutically effective quantity of an antibiotic and an antibody or an antigen-binding fragment thereof specific against a C. difficile LDH according to the present invention.

The antibiotic may be metronidazole, or the antibiotic may be a glycopeptide antibiotic e.g. vancomycin, ramoplanin, or teicoplanin

The infection may be due to C. difficile, which may be resistant to treatment by the antibiotic alone.

The invention will be further apparent from the following experiments which describe the isolation and characterization of C. difficile LDH.

### Experiments

The experiments detailed below demonstrate in immunoblotting experiments using fractionated C. difficile protein extracts and antisera obtained from patients infected with C. difficile that a protein of apparent molecular weight 36 kDa is recognised by a majority of patients. Using amino acid sequencing, this protein was determined to be lactate dehydrogenase (LDH). The gene encoding the C. difficile LDH protein was cloned, and then subcloned into a bacterial expression vector for expression of recombinant LDH protein. Purified recombinant LDH protein was fractionated by SDS-PAGE and 2D electrophoresis and immunoblotted prior to probing with antisera obtained from patients infected with C. difficile.

Unless stated otherwise, all procedures detailed herein were performed using standard protocols and following manufacturer's instructions where applicable. Standard protocols for various techniques including PCR, molecular cloning, manipulation and sequencing, and cell culturing, are described in texts such as McPherson, M. J. et al. (1991, PCR: A practical approach, Oxford University Press, Oxford), Sambrook, J. and Russell, D., (“Molecular Cloning: A Laboratory Manual”, Third Edition, Cold Spring Harbor Laboratory, Cold Spring Harbor Press, New York, 2001), Huynh and Davies (1985, “DNA Cloning Vol I —A Practical Approach”, IRL Press, Oxford, Ed. D. M. Glover), Sanger, F. et al. (1977, PNAS USA 74(12): 5463-5467), Harlow, E. and Lane, D. (“Using Antibodies: A Laboratory Manual”, Cold Spring Harbor Laboratory Press, New York, 1998),Harris, M. A. and Rae, I. F. (“General Techniques of Cell Culture”, 1997, Cambridge University Press, ISBN 0521 573645).

Reagents and equipment useful in, amongst others, the methods detailed herein are available from the likes of Amersham (www.amersham.co.uk), Boehringer Mannheim (www.boehringer-ingeltheim.com), Clontech (www.clontech.com), Genosys (www.genosys.com), Millipore (www.millipore.com), Novagen (www.novagen.com), Perkin Elmer (www.perkinelmer.com), Pharmacia (www.pharmacia.com), Promega (www.promega.com), Qiagen (www.qiagen.com), Sigma (www.sigma-aldrich.com) and Stratagene (www.stratagene.com).

Where “PMID” reference numbers are given for publications, these are the PubMed identification numbers allocated to them by the US National Library of Medicine, from which full bibliographic information and abstract for each publication is available at www.ncbi.nlm.nih.gov. This can also provide direct access to electronic copies of the complete publications, particularly in the case of e.g. PNAS, JBC and MBC publications.

The contents of each of the references discussed herein, including the references cited therein, are herein incorporated by reference in their entirety.

Methods

Samples

Serum samples and stool specimens were collected and stored at −20° C. and 4° C. respectively.

C. difficile Tox AB II Test

This kit was purchased from Tec Lab Inc., USA. 50 μl of liquid stool was added to 200 μl diluent and 100 μl of the mix was added to 50 μl of conjugate in a microtitre plate and incubated for 50 minutes at 35° C., followed by 4 washes. Substrates A and B were added to the plate, mixed and incubated for 10 minutes at room temperature. Stop solution was added the plate incubated for a further 10 minutes and the O.D. measured at 450 nm.

Culture and Identity

The positive specimens were cultured using C. difficile agar base (Oxoid) with C. difficile selective supplement, D-cycloserine and cefoxitin (Oxoid) and grown under anaerobic conditions for 48 hours. The identities of the positive specimens were confirmed using the enzymatic hydrolysis PRO test (Key Scientific Products, USA) and the latex agglutination test (Microscreen C. difficile, Microgen Bioproducts Ltd, UK).

Antigen Preparation

C. difficile NCTC 11204 was cultured under anaerobic conditions in medium containing 2% (w/v) protease peptone and 1% (w/v) yeast extract at 37° C. for 48 hours. The cells were harvested by centrifugation and washed 3 times in sterile saline. The cell paste was then placed into the Bio X-presser cell disintegrator (LKB Instruments Sweden) and left at −20° C. overnight prior to crushing. The samples were crushed washed and visualised by SDS-PAGE.

Sample Visualisation and Protein Identification

SDS-PAGE

Plates were assembled according to the manufacturer's instructions and a resolving gel (25 ml 30% acrylamide/bis, 14.06 ml separating gel buffer (24.22 g Tris base, made up to 100 ml with distilled water (pH 8.8), 750 μl 10% SDS, 35.2 ml distilled water, 375 μl 10% ammonium persulphate, 37.5 μl TEMED) was poured. Once set, a stacking gel (3.6 ml 30% acrylamide/bis, 7.3 ml stacking gel buffer (6.05 g Tris base made up to 100 ml with distilled water (pH 6.8), 300 μl 10% SDS, 19 ml distilled water, 300 μl 10% ammonium persulphate, 30 μl TEMED), was poured, a comb inserted and centred. The gel was left to set, the comb removed and the wells washed with distilled water. The gel was placed into the tank holder and into the tank. The samples (10 μl of C. difficile) were added to each well and electrophoresis buffer (18.96 g Tris base, 12 g glycine, 3 g SDS, 3 L distilled water) layered on top. The tank filled with electrophoresis buffer, the water turned on and run at a constant current of 80-100 mA for 1-2 hours.

Protein Visualisation

Protein bands were visualised by fixing the gels overnight at 4° C. in 100 ml methanol, 20 ml glacial acetic acid followed by silver staining using the OWL Silver Staining Kit (NBS Biologicals, Huntingdon).

Western Blotting

Gel holders were assembled according to the manufacture's instructions forming a sandwich as follows: Scotchbrite, 2× filter paper, nitrocellulose/PVDF, gel, 2× filter paper, Scotchbrite (the Scotchbrite, filter paper, and nitrocellulose/PVDF were all pre-soaked in transblotting buffer (9.07 g Tris base, 43.2 g glycine, 3 L distilled water, 750 ml methanol). The holders were slotted into the tank and run at 100A for 45 minutes.

Sera were available from 24 patients with clinical C. difficile colitis and a positive toxin assay on faeces, and from 20 patients with no evidence of colitis and a negative toxin assay. The sera were added to the transblotted antigen strips and incubated with shaking at room temperature for 2 hours. Five 5 minute washes were followed by a 1 hour incubation with anti-human IgG, IgA or IgM conjugated with alkaline phosphatase. The strips were washed prior to visualisation using BCIP/NBT.

Protein Sequencing

The appropriate band was transblotted onto PVDF membrane and direct amino acid sequencing was performed using standard methodologies.

Library Construction and Analysis of Clones

Genomic Library Construction

C. difficile NCTC 11204 was grown under anaerobic conditions overnight at 37° C. in 10 ml LB broth. The bacteria were harvested by centrifugation, and the pellet was washed twice in sterile saline and resuspended in 1 ml TE buffer. 1 μl lysozyme was added to the cells followed by an incubation at 37° C. for 15 minutes. 20 μl of proteinase K and 50 μl of 10% SDS was added to the mixture, which was inverted to mix and left at 55° C. for 45 minutes. The mixture was pulsed in a microfluge, the supernatant was transferred to a tube containing an equal volume of phenol:chloroform:isoamylalcohol (25:25:1) and mixed by inversion for 10 minutes to precipitate protein. This was followed by centrifugation at 12000×g for 10 minutes. The upper aqueous DNA-containing layer was removed and the above step repeated. The DNA was precipitated by mixing the aqueous layer with an equal volume of isopropanol and placing the mixture at room temperature for 15 minutes, followed by centrifugation at 12,000 g for 20 minutes. The pellet containing the bacterial DNA was washed in ice cold 70% ethanol to remove excess salt, and resuspended in 200 μl ultrapure water.

Partial Digestion of Bacterial Genomic DNA

A restriction digest was set up containing Hind III, used according to the manufacturer's instructions, and 5 μl aliquots were collected at time intervals and deactivated by heating to 65° C. for 10 minutes. The DNA samples were ethanol precipitated and visualised by agarose gel electrophoresis. A fragment corresponding to 2-5 Kb was excised from the gel and cleaned up using the Geneclean protocol (Anachem, UK). The DNA was eluted in 20 μl sterile ultrapure water and stored at −20° C.

Purification of Plasmid DNA

5 ml LB broth containing 100 μg/ml ampicillin was inoculated with E. coli containing the plasmid pTZ18R and grown overnight at 37° C. in a shaking platform incubator. The bacterial cells were harvested by centrifugation and plasmid DNA was isolated and purified using the QIAprep Spin Miniprep Kit (QIAgen).

Plasmid Digestion, Dephosphorylation and Ligation

pTZ18R DNA was digested with Hind III and 5′ phosphate groups were removed by adding 4 μl of 10× alkaline phosphatase buffer (Promega) and 1 μl alkaline phosphatase (Promega) to 35 μl of sample, followed by an incubation at 37° C. for 2 hours. The sample was visualised on an agarose gel and cleaned up using the Geneclean kit, as described previously. The partially digested genomic DNA was ligated with pTZ18R in a small volume at 4° C. overnight using T4 DNA ligase (Promega) according to the manufacturers instructions.

Transformation

Plasmid DNA was transformed by electroporation (200 ohms/25 μF 2.5 KV) into the following bacterial strains:

E. coli JM109,

E. coli ldh—DC1368 (lactate dehydrogenase deficient) IahA: Kanpfi,com (Gupta, S., and Clark, D. P., 1989, J. of Bacteriology, 171, p3650-3655)

E. coli W1485 (wild type).

The transformed bacterial cells were plated onto M9 media and incubated at 37° C. overnight in both aerobic and anaerobic conditions.

DNA Sequencing

Individual clones were sequenced using a MegaBACE 1000 DNA Sequencer (Amersham Pharmacia Biotech). Plasmid DNA (100-500 ng) was added to 8 μl DYEnamic ET terminator reagent premix and lμl (5 μM) M13 forward (SEQ ID NO: 5) or M13 reverse primer (SEQ ID NO: 6) in a total volume of 20 μl. Cycling conditions were 30 cycles of 95° C., 20 seconds; 50° C., 15 seconds; and 60° C., 60 seconds. Unincorporated dye terminators were removed by ethanol precipitation and the DNA resuspended in 20 μl loading buffer. The reactions were loaded onto the DNA Sequencer using injection parameters of 2 KV for 30 seconds and electrophoresis at 6 KV for 200 minutes.

Subcloning of ldh Gene

The ldh gene was reamplified from the partially digested DNA which had been subcloned into the pGEM-T easy vector (Promega, Southampton, UK) using the polymerase chain reaction (PCR) under the following conditions; 1 μg DNA, 25 pmol of each of L1 forward (SEQ ID NO: 3) and L2 reverse (SEQ ID NO: 4) primers, 200 μM dNTPs, 10 mM Tris-HCl pH 8.8, 50 mM KCl, 1.5 mM MgCl₂ and 5 units Taq polymerase. Cycling conditions utilised an initial denaturing step of 5 minutes at 94° C. followed by 25 cycles of 94° C., 60 seconds; 55° C., 60 seconds; and 72° C., 60 seconds, followed by a final extension step of 7 minutes at 72° C., and 4° C. hold. The samples were visualised by agarose gel electrophoresis. The ldh gene was subcloned into the pBAD-TOPO vector (Invitrogen) as follows; 2 μl of the Genecleaned PCR product (above) was added to 1 μl pBAD-TOPO vector and 1 μl of the supplied salt solution in a final reaction volume of 5 μl and incubated at room temperature for 5 minutes. E. coli TOP10-F′ competent cells were transformed by heat-shock using 2 μl of the final reaction and after incubation at 37° C. for 1 hour in SOC medium, the cells were plated onto dry LB agar plates supplemented with 100 μg/ml ampicillin, and the plates were incubated in an inverted position overnight at 37° C.

Positive Clones

Positive clones were amplified by PCR. One colony was added to a PCR master mix containing 50 pmol of each of pBAD forward (SEQ ID NO: 7) and L1 reverse primer, 200 μM dNTPs, 10 mM Tris-HCl pH 8.8, 50 mM KCl, 1.5 mM MgCl₂ and 5 units Taq polymerase. Cycling conditions utilised an initial denaturing step of 5 minutes at 94° C. followed by 25 cycles of 94° C., 60 seconds; 55° C., 60 seconds; and 72° C., 60 seconds, followed by a final extension step of 7 minutes at 72° C., and 4° C. hold. The samples were visualised by agarose gel electrophoresis.

Expression of Recombinant LDH Protein

Each positive transformant was cultured with shaking in 10 ml LB supplemented with 100 μg/ml ampicillin at 37° C. overnight. 100 μl of the culture was used to inoculate fresh 10 ml broths the next morning which were subsequently grown at 37° C., with shaking until an OD₆₀₀ of 0.5 was obtained. Protein expression was induced by the addition of 20% (w/v) L-arabinose followed by further incubation at 37° C. for 4 hours. The cells were pelleted by centrifugation at 4000×g for 15 minutes and resuspended in 100 μl SDS-PAGE buffer. The samples were heated to 70° C. for 5 minutes, and 5 μl fractionated on a gel and transblotted as described previously. Protein bands were detected using a 1:5000 dilution of anti-V5 epitope tag antibody, followed by an incubation with an anti-mouse alkaline phosphatase conjugate and BCIP/NBT stain.

Purification of Recombinant Protein

A 50 ml culture was grown and protein expression induced as described above. Recovered cells were lysed using the Bio X-Presser cell disintegrator (LKB Instruments). The protein was purified using the ProBond Purification System (Invitrogen), used according to the manufacturers instructions. The protein was eluted by either sequentially increasing the imidazole concentration using 5 ml of 50 mM, 200 mM, 350 mM and 500 mM imidazole elution buffers consecutively and collecting 1 ml fractions or applying 5 ml of the native pH elution buffer and collecting 1 ml fractions. Fractions were analysed by SDS-PAGE and Western blotting, essentially as described previously.

SDS-PAGE and Western Blotting Using Patient Sera

Fractions containing recombinant protein were run on an SDS-PAGE gel and immunoblotted using sera from 10 patients with confirmed C. difficile infections and 4 control sera.

2D-gel Electrophoresis and Western Blotting Using Patient Sera

Sample preparation

C. difficile cells suspended in 10 mM PBS were sonicated on ice for five one minute bursts. The resulting cell lysate was centrifuged at 13000 rpm for 5 minutes and 300 μof the supernatant was precipitated in 20 ml 10% trichloroacetic acid/20 mM DTT in cold acetone for 45 minutes. The proteins were recovered by centrifugation at 13000 rpm for 5 minutes and the resulting pellet was washed with cold acetone containing 20 mM DTT. The centrifugation and washing step was repeated two more times, and the proteins were recovered by centrifugation as before, prior to fractionation by 2D gel electrophoresis.

2D-gel Electrophoresis

The protein pellet was dissolved in sample rehydration solution and diluted with the same solution to an appropriate concentration for isoelectric focusing. Isoelectric focusing was performed using a Zoom®IPGRunner™System (Invitrogen Ltd, Carlsbad, Calif., USA) over a non-linear pH range of 3-10 (7 cm) for a total of 1700 Vh, loading approximately 15 μg of protein on each strip. Prior to the second dimension separation the strips were equilibrated for 15 minutes in equilibration buffer containing 65 mM DTT and then in the same buffer containing 125 mM iodoacetamide for another 15 minutes.

The second dimension separation was carried out using NuPage 4-12% Bis-TrisZoom gel (Invitrogen Ltd, Carlsbad, Calif., USA) gels. The proteins were transblotted onto Invitrolon PVDF membrane (Invitrogen Ltd, Carlsbad, Calif., USA) and blocked for 1 hour in 10 mM PBS containing 5% skimmed milk/0.1% Tween 20.

Western Blotting

To identify the position of LDH, the membrane was incubated for one hour with a polyclonal anti-LDH-horse radish peroxidase antibody conjugate (Abcam Limited, Cambridge,UK), which had been raised against LDH from rabbit muscle. The antibody was diluted (1:500) in 10 mM PBS/0.1% Tween 20 (PBS-T). After washing in PBS-T the blot was developed using SigmaFast™3,3′-diaminobenzidine tablets (DAB).

Patient antisera, which were known to be immuno-reactive to C. difficile proteins of 3040 kDa (as determined by one dimensional SDS-PAGE and immunoblotting), and therefore were potentially immuno-reactive to LDH, were selected to probe the two-dimensional blots in the same way as described above for the identification of LDH. The membranes were incubated for 50 minutes in serum diluted (1:20) in PBS-T. After washing the blots in PBS-T, primary antibodies were detected using a secondary anti-human IgG-alkaline phosphatase conjugate (Fc specific). Primary/secondary antibody complexes were detected using SigmaFast™ 5-bromo-4-chloro-3-indolyl phosphate/nitro blue tetrazolium tablets.

Results

Samples

24 of the samples tested were positive by C. difficile TOX A/B II test, giving a yellow (positive) reaction. Identification was confirmed by colonial and gram morphology (gram positive Bacillus with large oval sub terminal spore on blood agar), a positive reaction to the enzymatic hydrolysis PRO test (a dark pink to red appearance) and the latex agglutination test gave visible clumps within 2 minutes indicating the presence of C. difficile. Whole antigen was provided from cultures of both clinical isolates and standard (National Collection of Type Cultures (NCTC) 11204, PHLS, London, UK) strains of C. difficile.

SDS-PAGE

OWL silver staining of C. difficile whole cell extracts showed protein bands with molecular sizes between 20 and 200 kDa.

Western Blotting

IgG blotting of whole cell extracts of C. difficile using patient and control serum samples resulted in detection of a protein of apparent molecular weight 36% kDa which had statistically significant differences between the two groups. This protein was detected in 21 of the 24 patient samples as opposed to 7 of the 20 control samples (X²=12.37, P value 0.001). Other immunodominant antigens were apparent at 43, 55 and 70 kDa (Table 1).

TABLE 1
Details of the antibody responses (IgM, IgG and IgA)
against immunodominant antigens of C. difficile.

| Antigen | | | | | | |
|---|---|---|---|---|---|---|
| Apparent | | | | | | |
| Molecular | Control (n = 20) | | | Colitis Patients (n = 24) | | |
| Weight (kDa) | IgM | IgG | IgA | IgM | IgG | IgA |
| 36 | 1 | 7 | 0 | 4 | 21 | 3 |
| 43 | 2 | 11 | 0 | 0 | 17 | 0 |
| 55 | 2 | 7 | 0 | 4 | 11 | 6 |
| 70 | 2 | 9 | 0 | 8 | 13 | 6 |

Protein Sequencing

The first 10 amino acid residues from the 36 kDa band were obtained by N-terminal sequencing, and are shown in Table 2. This amino acid sequence was entered into the BCM Search Launcher (www.searchlauncher.bcm.tmc.edu/) which returned matches showing 60% homology with ldh, as shown in Table 3.

TABLE 2
N-terminal sequence analysis results of the protein sequencing

| 1 | 2 | 3 | 4 | 5 | 6 | 7 | 8 | 9 | 10 |
|---|---|---|---|---|---|---|---|---|---|
| M | K | I | L | V | F | G | A | R | D |
| Met | Lys | Ile | Leu | Val | Phe | Gly | Ala | Arg | Asp |

TABLE 3
Comparison of the first 10 amino acids of the
identified 36 kDa protein (using BLAST)

| Protein | Number of base pairs | % Identity | Function |
|---|---|---|---|
| D-LDH | 30 | 60 | Lactate dehydrogenase |
| D-LDH | 331 | 60 | Lactate dehydrogenase |
| D-LDH | 331 | 60 | Lactate dehydrogenase |

Genomic Library

The genomic DNA was partially digested with Hind III to produce DNA fragments ranging from 2-5 Kbp. The partially digested DNA was ligated into pTZ18R and transformed into E. coli. Recombinant clones were screened for growth and sequenced. Primers derived from this were used to PCR the ldh gene from the library prepared in pGEM-T easy (Promega) and transformed into E. coli JM109. Recombinant clones were confirmed by PCR and selected for sequencing.

DNA Sequencing

Sequencing of both products revealed a open reading frame containing the ldh gene. The insert was 950 base pairs long and encoded 310 amino acids. This resulted in partial matches (29-42% homology) with 7 other LDH proteins (including Lactobacillus spp., Pediococcus acidilactici, Leuconostoc mesenteroides and E. coli).

SDS-PAGE and Western Blotting

Recombinant LDH protein was recognised by antibodies contained within eight of the ten patient antisera. In contrast, the recombinant protein was not detected in Western blots using any of the four control sera.

2D gel Electrophoresis and Immunoblotting

Using a commercial anti-lactate dehydrogenase antibody it was possible to identify the LDH protein on the 2D blot. Only two intense spots were visualised by the antibody, of which only one exhibited the estimated molecular weight (approximately 34 kDa) and the estimated isoelectric point (calculated to be 4.95). Of the three patient sera that were analysed, all of them contained antibodies to LDH. One patient's antibodies were nearly exclusively reactive to LDH.

### SUMMARY

These experiments describe the identification of LDH as a protein which is recognised by antibodies within sera obtained from patients infected with C. difficile.

### EXAMPLES

To treat a patient infected with C. difficile, antibodies or antigen-binding fragments thereof specific against the bacterial LDH protein or epitopes derived from the LDH protein are administrated to the patient orally or intravenously. An appropriate dosage is readily determined using dose response assays.

A patient is treated using a therapeutically effective combination of antibody and a glycopeptide antibiotic, for example vancomycin. The glycopeptide antibiotic is administered orally, for example as a tablet or capsule. Appropriate dosages of glycopeptide antibiotics are well known to a person skilled in the art. The antibiotic is orally administered in conjunction with the antibody, for example within the same formulation, or the antibiotic is administered at approximately the same time as the administration of the antibody. Antibodies or antigen-binding fragments thereof specific against the bacterial LDH protein or epitopes derived from the LDH protein are administrated orally or intravenously. An appropriate dosage is readily determined using dose response assays.

To vaccinate a person to decrease or eliminate susceptibility to C. difficile infection, or stimulate a patients immune response against the bacterium, a vaccine is administered to a patient, for example suffering from CDAD or C. difficile colitis, who may or may not be capable of mounting an immune response to the bacterium. The vaccine comprises a bacterial extract, or recombinant LDH protein, or epitopes derived from the protein, or combinations thereof. For those patients unable to mount an immune response to the bacterium, a vaccine comprising LDH or epitopes thereof, represents a means by which the patients immune system is stimulated to mount an appropriate response. Antibody responses are determined by monitoring the levels of anti-LDH antibodies in a sample from the patient. The vaccine comprises an adjuvant to enhance or improve the immunogenicity of the vaccine components. Patients whose immune responses are weak are rechallenged such that multiple doses of the vaccine are administered over a set period of time.

C. difficile is detected in a patient sample by taking an e.g. blood sample from a patient, and contacting it with an antibody or an antigen-binding fragment thereof specific against C. difficile LDH. For example, SDS-PAGE and Western blotting using specific anti-LDH antibodies is used to determine if C. difficile is present within the sample—a positive reaction as determined by Western blotting, i.e. the detection of a protein corresponding to the molecular weight of LDH indicates the presence of C. difficile within the sample.

Antibodies specific for C. difficile antigens are detected in a sample taken from a patient, for example using SDS-PAGE and Western blotting. For example, recombinant bacterial LDH, antigenic fragments thereof, or a bacterial extract are fractionated by SDS-PAGE and probed with patient antisera. A positive reaction as determined by Western blotting, i.e. the detection of a protein corresponding to the molecular weight of LDH indicates the presence of anti-C. difficile antibodies within the patient sample.

The antibody or an antigen-binding fragment thereof specific against C. difficile LDH is contained within a diagnostic test kit. Similarly, a diagnostic test kit contains recombinant bacterial LDH, fragments thereof, or a bacterial extract.

The antibody or an antigen-binding fragment thereof specific against C. difficile LDH is contained within a pharmaceutical pack for the treatment of a Clostridium difficile infection. The pharmaceutical pack comprises a therapeutically effective quantity of a glycopeptide antibiotic e.g. vancomycin.

1. A Clostridium difficile lactate dehydrogenase comprising the amino acid sequence of SEQ ID NO: 2, or an amino acid sequence exhibiting at least 70, 80, 90, 95, 96, 97, 98, 99, or 99.5% identity with the amino acid sequence of SEQ ID NO: 2.

2. An isolated nucleic acid molecule encoding the Clostridium difficile lactate dehydrogenase as claimed in claim 1.

3. An isolated nucleic acid molecule according to claim 2, comprising the nucleotide sequence of SEQ ID NO: 1.

4. A nucleic acid vector comprising the nucleic acid molecule of claim 2.

5. A host cell containing the vector of claim 4.

6. A process for producing a polypeptide comprising the Clostridium difficile lactate dehydrogenase according to claim 1, the process comprising culturing the host cell of claim 5 under conditions sufficient for the production of said polypeptide, and recovering said polypeptide.

7. A vector according to claim 4, wherein said vector is selected from the group consisting of a plasmid, a virus, and a bacteriophage.

8. A vector according to claim 7, wherein said isolated nucleic acid molecule is inserted into said vector in proper orientation and correct reading frame such that a polypeptide comprising the Clostridium difficile lactate dehydrogenase may be expressed by a cell transformed with said vector.

9. A vector according to claim 8, wherein said isolated nucleic acid molecule is operatively linked to a promoter sequence.

10. An antibody or an antigen-binding fragment thereof specific against a Clostridium difficile lactate dehydrogenase according to claim 1.

11. An antibody or an antigen-binding fragment thereof according to claim 10 for use in a method of treatment or diagnosis of the human or animal body.

12. (canceled)

13. A medicament, comprising a therapeutically effective quantity of an antibiotic and an antibody or an antigen-binding fragment thereof specific against a Clostridium difficile lactate dehydrogenase according to claim 1.

14. A medicament according to claim 13 for use in a method of treatment or diagnosis of the human or animal body.

15. A medicament according to claim 13, being for the treatment of a Clostridium difficile infection.

16. A method of treatment of a Clostridium difficile infection, comprising the step of administering to a patient in need thereof a therapeutically effective quantity of an antibody or an antigen-binding fragment thereof specific against a Clostridium difficile lactate dehydrogenase according to claim 1.

17. A method of treatment according to claim 16, further comprising the step of administering to a patient a therapeutically effective quantity of an antibiotic.

18. A diagnostic test method for detecting the presence in a sample of a Clostridium difficile lactate dehydrogenase according to claim 1, comprising the steps of:
i) contacting said sample with an antibody or an antigen-binding fragment thereof specific against said Clostridium difficile lactate dehydrogenase;
ii) detecting any antibody-antigen binding reaction; and
iii) correlating the results of detection step (ii) with the presence of said Clostridium difficile lactate dehydrogenase in said sample.

19. A diagnostic test method for detecting the presence in a sample of antibody specific against a Clostridium difficile lactate dehydrogenase according to claim 1 comprising the steps of:
i) contacting said sample with said Clostridium difficile lactate dehydrogenase;
ii) detecting any antibody-antigen binding reaction; and
iii) correlating the results of detection step (ii) with the presence of antibody specific against said Clostridium difficile lactate dehydrogenase in said sample.

20. A diagnostic test method according to claim 18, the sample being a sample from a patient.

21. A diagnostic test kit for performing a diagnostic test method according to claim 18.

22. A pharmaceutical pack for the treatment of a Clostridium difficile infection, comprising a therapeutically effective quantity of an antibiotic and an antibody or an antigen-binding fragment thereof specific against a Clostridium difficile lactate dehydrogenase according to claim 1.

23. A medicament, method of manufacture, method of treatment, diagnostic test method, diagnostic test kit, or pharmaceutical pack according to claim 13, 16, 18, 19, 21, or 22, said antibiotic being vancomycin, ramoplanin, teicoplanin, or metronidazole.

24. A medicament, method of manufacture, method of treatment, diagnostic test method, diagnostic test kit, or pharmaceutical pack according to claim 13, 16, 18, 19, 21, 22, said infection being due to Clostridium difficile.

25. A medicament, method of manufacture, method of treatment, diagnostic test method, diagnostic test kit, or pharmaceutical pack according to claim 13, 16, 18, 19, 21 or 22, said bacterium being resistant to treatment by said antibiotic alone.

26. A diagnostic test method according to claim 19, the sample being a sample from a patient.

27. A diagnostic test method for performing a diagnostic test method according to claim 19.


SEQUENCE LISTING (8 sequences)

SEQ ID NO: 1 - DNA, 975 bp, Clostridium difficile
  CDS (40)..(972)
attaccataataatttaataattacaggagggtattgatatgaaaatactagtatttggagcacgcgattatgaagaaccagtaataaaaaaatggtctgaagaacataaggatgttcaagtggatatttatcctgaaaacatgactgaagaaaatgtagttaaagctaaagggtatgatggtatatctatacaacaaactaactatatagataatccttatatttatgaaactttaaaagatgctggggttaaagttatagcttcaagaactgcaggggttgacatgatacattttgatttagttaatgaaaatggacttatcgttacaaacgttcctgcttattcacctaatgcaatagctgaattagctgttactcaagctatgaaccttttaagaaagactcctctagtaaagaaaaaagtctgtgaaggtgattaccgttggatagctgaacttcttggaacagaagttagatctattacagttggtgttataggtacaggaaaaataggtgctacttctgcaaaattattcaaaggcctaggagctaatgtaattgcatatgaccaatatccaaatagtgatttaaacgatatattaacttacaaagattctttagaagaccttctaaaagaagctgacgttataacattacatactcctttacttgaaggaacaaaacatatgataaataaagatactctagctataatgaaggatggagcttacatagtaaatactgcccgtggtggtttaattgatacaggggatttaatagaagcactagactcaggaaaaattagagctgctgcccttgatacatttgaaactgaaggattgttcttaaacaaaaaaatgaatcctggagaattaactgacccagaaataaataaacttctttctatggaacaagttatattcactcatcatcttggtttcttcactagtacagcgattgaaaatatagtttaa

SEQ ID NO: 2 - protein, 311 aa, Clostridium difficile
MKILVFGARDYEEPVIKKWSEEHKDVQVDIYPENMTEENVVKAKGYDGISIQQTNYIDNPYIYETLKDAGVKVIASRTAGVDMIHFDLVNENGLIVTNVPAYSPNAIAELAVTQAMNLLRKTPLVKKKVCEGDYRWIAELLGTEVRSITVGVIGTGKIGATSAKLFKGLGANVIAYDQYPNSDLNDILTYKDSLEDLLKEADVITLHTPLLEGTKHMINKDTLAIMKDGAYIVNTARGGLIDTGDLIEALDSGKIRAAALDTFETEGLFLNKKMNPGELTDPEINKLLSMEQVIFTHHLGFFTSTAIENIV

SEQ ID NO: 3 - DNA, 20 bp, Clostridium difficile
atgaaaatactagtatttgg

SEQ ID NO: 4 - DNA, 21 bp, Clostridium difficile
ttaaactatattttcaatcgc

SEQ ID NO: 5 - DNA, 17 bp, Clostridium difficile
gttttcccagtcacgac

SEQ ID NO: 6 - DNA, 17 bp, Clostridium difficile
caggaaacagctatgac

SEQ ID NO: 7 - DNA, 20 bp, Clostridium difficile
atgccatagcatttttatcc

SEQ ID NO: 8 - protein, 10 aa, Clostridium difficile
MKILVFGARD